(19) United States
(12) Patent Application Publication
(10) Pub. No.: US 2002/0076423 A1
(43) Pub. Date: Jun. 20, 2002

(54) Cosmetic or dermatological formulations for the care and cooling of the skin after sunbathing

(76) Inventors: Rainer Kropke, Schenefeld (DE); Boris Syskowski, Hamburg (DE); Birgit Groteluschen, Hamburg (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 09/907,031

(22) Filed: Jul. 17, 2001

(30) Foreign Application Priority Data
Jul. 25, 2000 (DE) 100 36 051.3

(51) Int. Cl. A61K 7/42; A61K 7/00; A61K 31/722

(52) U.S. Cl. 424/401; 424/59; 514/55

(57) ABSTRACT

Cosmetic or dermatological formulations with a content of

a) at least one volatile substance which has an evaporation number of less than 30,

b) at least one chitosan and

c) at least one lecithin

and the use thereof.

The present invention relates to cooling cosmetic or dermatological formulations, in particular cosmetic or dermatological formulations which care for the skin in a targeted manner after sunbathing and which reduce the secondary reactions of the skin to the effect of UV radiation.

In addition to the positive effects of sunlight, such as general wellbeing, the formation of vitamin D3 and the treatment of acne, there are also negative effects which must be counteracted.

If the skin is exposed for too long to the sun or to a source of artificial rays, after a latency period of from 2 to 3 hours, a reddening of the skin, starkly demarcated from the unradiated skin, arises (erythema solar). Sunburn which has arisen in this way is differentiated as follows:

1st degree: Erythema (reddening, feeling of warmth) subsides again after 2 to 3 days and disappears with a simultaneous increase in pigmentation,

2nd degree: Blister formation blisters form on the skin with burning and itching, and sections of the epidermis are shed

3rd degree: Cell damage deep cell damage occurs, the body reacts with fever, large sections of the epidermis are shed.

The 2nd and 3rd degrees are also referred to as dermatitis solar.

The formation of the erythema is dependent on the wavelength. The erythema region of UV-B is between 280 nm and 320 nm.

Approximately 90% of the ultraviolet radiation which reaches the earth consists of UV-A rays with a wavelength between 320 nm and 400 nm. Whereas UV-B radiation varies widely depending on numerous factors (e.g. season and time of day or latitude), UV-A radiation remains relatively constant from day to day irrespective of seasonal and diurnal or geographic factors. At the same time, most of the UV-A radiation penetrates into the living epidermis, while about 70% of the UV-B rays are retained by the horny layer.

For a long time it has incorrectly been assumed that the long-wavelength UV-A radiation has only a negligible biological effect and that, correspondingly, the UV-B rays are responsible for most photodamage to the human skin. In the meantime, however, numerous studies have proven that UV-A radiation is far more hazardous than UV-B radiation with regard to the triggering of photodynamic, specifically phototoxic, reactions and chronic changes in the skin. The harmful effect of UV-B radiation can also be intensified by UV-A radiation.

Since the contributions of the various wavelength ranges of UV light to light-induced skin changes have not been fully explained, it is nowadays increasingly assumed that preventive protection both against UV-A and also against UV-B rays, for example by applying light protection filter substances in the form of a cosmetic or dermatological formulation to the skin, is of fundamental importance. Cosmetic or dermatological compositions are intended, when applied to the skin in a thin layer, to protect it against the negative effects of solar radiation.

Sunbathing is regarded by most people as pleasurable, and the disadvantageous consequences are initially not taken into account. However, in recent years, knowledge about the negative effects of excessively intensive solar irradiation has emerged, for which reason more and more strongly protecting sunscreens are used.

Sunburn or photoerythema are the acute manifestations of the effect of light. As well as the effects of UV rays which have already been described, a secondary reaction in the skin also leads to reduced sebum production and drying-out of the skin. To alleviate and care for photodamaged skin, it is therefore possible to use specific active ingredients, such as, for example,

refatting agents and humectants,

inflammation-alleviating and cooling substances,

local anesthetic substances and/or

disinfecting substances, in order to prevent possible skin infections.

Aftersun preparations are intended to cool the skin after sunbathing and to improve its ability to retain moisture, the provision of the cooling effect playing a central role. This cooling effect is generally achieved using large amounts of ethanol which spontaneously evaporates upon distributing the formulation on the skin.

However, a disadvantage of these prior art formulations is that it is not possible to achieve long-term cooling since the ethanol evaporates very rapidly and the cooling effect which arises as a result is accordingly only of short duration.

The prior art recognizes further preparations which, applied to the skin or mucous membranes, are intended to have a moisturizing and cooling action. For example, in the literature, ionic compounds, in particular ammonium salts, are described as cooling agents. As cooling preparations, isopropanol gels with an addition of camphor and menthol are also used widely, and, in general, essential oils, primarily camphor and menthol, but also derivatives thereof (e.g. menthyl lactate or menthyl 3-hydroxybutyrate) are often incorporated into cooling compositions.

Menthol, camphor and derivatives thereof, but also other essential oils lower the sensitivity threshold of the neuronal cold receptors and thus bring about a cold feeling. However, they also frequently bring about an increase in circulation which, in contrast, brings about a warm feeling. The use of these substances, in particular on irritated skin, is in any case problematical. Moreover, many of these compounds are poorly soluble in water. As a consequence, their use is limited to a few cosmetics and dermatological agents.

An object of the present invention was therefore to find cosmetic or dermatological preparations which do not have the disadvantages of the prior art and which cool, in particular photodamaged, skin for a long period.

It was surprising and herein lies the attainment of these objects that cosmetic or dermatogical formulations with a content of

a) at least one volatile substance which has an evaporation number of less than 30,

b) at least one chitosan and

c) at least one lecithin

would overcome the disadvantages of the prior art.

The formulations according to the invention are satisfactory preparations in every respect and are notable for a long-lasting cooling effect. It had not been foreseen by the person skilled in the art that the formulations according to the invention

would better care for photodamaged skin,

would better reduce the secondary reactions of the skin to the effect of UV radiation,

would better calm skin irritated from sunbathing,

would more quickly alleviate mild sunburn,

would have a better action than moisture-donating preparations,

would be easier to formulate,

would better promote skin smoothing,

would be characterized by better care action,

would better serve as vehicles for cosmetic and medicinal-dermatological active ingredients and

would have better sensory properties, such as, for example, ability to be distributed on the skin or the ability to be absorbed into the skin

than the preparations of the prior art.

The invention is of course not limited to preparations which are applied after sunbathing, but naturally includes all cosmetic and dermatological applications in which a cooling action or a cooling effect could be desired or advantageous.

The invention therefore also provides for the use of

1. at least one chitosan and

2. at least one lecithin

in a cosmetic or dermatological formulation for delaying the evaporation of volatile substances which have an evaporation number of less than 30, following application of the formulation to the skin or the scalp.

Volatility is generally understood as meaning the evaporation behavior or the evaporation rate of substances, specifically liquids. Since the volatility of real liquids is dependent on many parameters, it can not really be calculated theoretically. To assess the volatility, therefore, use is made of measurement values determined empirically. The evaporation number (EN) is a measurement value for the volatility. A related characteristic value is the evaporation coefficient (EC). The evaporation number is understood as meaning the quotient of the evaporation time of the liquid being tested and that of diethyl ether as comparison liquid; the test temperature is 293±2 K, and the relative atmospheric humidity should be 65%±5% during measurement (cf. DIN 53170: 1991-08; 53249; 1995-01).

For the purposes of the present invention, volatile substances are accordingly, for example, ethanol (EN=8.3), isopropanol (EN=1.7), n-hexane (EN=8.3), n-heptane (EN=3.7) and the like.

For the purposes of the present invention, volatile substances whose evaporation number is between 1 and 30, in particular those whose evaporation number is between 1 and 20, are particularly advantageous.

Chitosan is characterized by the following structural formula:

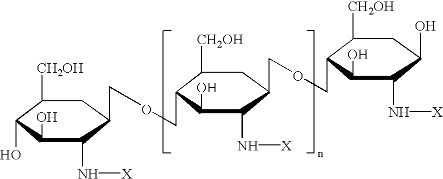

in which n assumes values up to about 2000 and X is either the acyl radical or hydrogen. Chitosan is formed by deacetylation and partial depolymerization (hydrolysis) of chitin, which is characterized by the structural formula

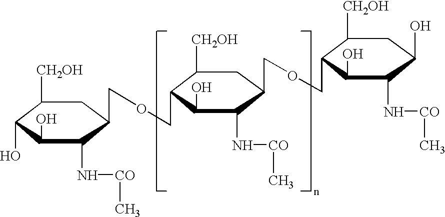

The use of chitosan in cosmetics is known per se. Thus, for example, chitosan is a raw material frequently used in haircare. It is suitable, more so than the chitin on which it is based, as a thickener or stabilizer and improves the adhesion and water resistance of polymeric films. Moreover, chitosan can also be used as a moisturizer, i.e. it is assumed that chitosan is able to reduce the moisture loss from the horny layer (also called transepidermal water loss (TEWL)) and/or positively influence the hydration of the horny layer. A representative of the large number of literature references for the prior art is: H. P. Fiedler, “Lexikon der Hilfsstoffe für Pharmazie, Kosmetik und angrenzende Gebiete” [Lexicon of auxiliaries for pharmacy, cosmetics and related fields], third edition 1989, Editio Cantor, Aulendorf, p. 293, keyword “chitosan”.

According to the invention, preference is given to chitosans with a degree of deacetylation of at least 50%, particularly with a degree of deacetylation of more than 60%, in particular more than 80%. Of these, particular preference is given to those whose 1% strength aqueous solution has a maximum viscosity of 5000 mPas (Haake viscotester VT-02 at 25° C.).

For the purposes of the present invention, advantageous chitosans are, for example, those available under the trade name Chitosan L from Primex and those available under the trade names Natural Biopolymer Poly+L, Poly+V and Poly+M from Natural Biopolymer.

The emulsions according to the invention advantageously comprise 0.05 to 5% by weight of chitosan, preferably 0.5 to 2% by weight, in particular 1% by weight, in each case based on the total weight of the preparations.

Lecithins belong to the group of phosphatides (phospholipids). They are characterized by the general structure

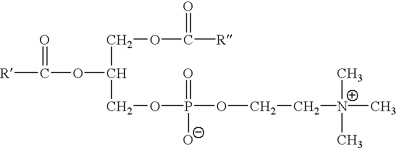

where R′ and R″ are typically unbranched aliphatic radicals having 15 or 17 carbon atoms and up to 4 cis double bonds.

The difference in the fatty acid radicals R′ and R″ gives rise to a large number of different lecithins. In the case of extraction from biological material, mixtures are always obtained. Lecithins are obtained, for example, from the egg of a chicken (egg lecithin) or from soybeans (soybean lecithin). Depending on their origin, the composition of the lecithins exhibits considerable differences. For example, a lecithin fraction from soybeans (the most widely used raw material) comprises, for example, palmitic acid, stearic acid, palmitoleic acid, oleic acid, linoleic acid and linolenic acid.

Lecithins are brownish-yellow, slightly hygroscopic, wax-like masses which are readily soluble in ether and often also in alcohol. In water, lecithins swell like a lyophilic colloid and then form a transparent, colloidal solution.

Lecithins are predominantly used as emulsifiers in the food industry in margarine, chocolate, bakery products and coatings. However, they are also used in cosmetic preparations.

For the purposes of the present invention, advantageous lecithins are, for example, those available under the trade names Sternpur PM, Sternpur E and Nathin 3KE from Stern.

It is advantageous according to the invention if the cosmetic or dermatological emulsions comprise 0.1 to 5% by weight, in particular 0.5 to 2.5% by weight, of one or more lecithins, in each case based on the total weight of the emulsions.

As well as comprising one or more oil phases, the cosmetic or dermatological formulations for the purposes of the present invention may additionally comprise one or more water phases and, for example, be in the form of W/O, O/W, W/O/W or O/W/O emulsions. Such emulsions may preferably also be a microemulsion, a Pickering emulsion or a sprayable emulsion.

The formulations according to the invention preferably also comprise anti-inflammatory substances, such as, for example, allantoin, α-bisabolol, pantothenic acid, panthenol, royal jelly, chamomile extracts, azulene or aloe vera extract, and unsaponifiable fractions of avocado or soybean oil and further substances which calm irritated skin. Further advantageous active ingredients are tannins, which have an astringent, anti-inflammatory and/or antisecretory action.

Moreover, the formulations according to the invention can also advantageously comprise dihydroxyacetone or nut extracts, and other substances which are said to achieve tanning.

The cosmetic and/or dermatological formulations according to the invention can have the customary composition and be used, in particular, for the treatment and care of the skin and/or the hair after sunbathing and as a make-up product in decorative cosmetics. Accordingly, the formulations according to the invention can, depending on their formulation, be used, for example, as skin protection cream, cleansing milk, sunscreen lotion, nourishing cream, day cream or night cream etc. In some instances, it is possible and advantageous to use the formulations according to the invention as a basis for pharmaceutical formulations. Preference is given in particular to cosmetic and dermatological formulations present in the form of an aftersun skincare product.

For use, the cosmetic and dermatological formulations according to the invention are applied to the skin and/or the hair in a sufficient amount in the manner customary for cosmetics.

The cosmetic and dermatological formulations according to the invention may comprise cosmetic auxiliaries such as those customarily used in such preparations, e.g. preservatives, bactericides, perfumes, antifoams, dyes, pigments which have a coloring effect, thickeners, moisturizers and/or humectants, fats, oils, waxes or other customary constituents of a cosmetic or dermatological formulation, such as alcohols, polyols, polymers, foam stabilizers, electrolytes, organic solvents and/or silicone derivatives, and moisturizers.

Moisturizers is the term used to describe substances or mixtures of substances which, following application or distribution on the surface of the skin, impart to cosmetic or dermatological preparations the property of reducing the moisture loss by the horny layer (also called transepidermal water loss (TEWL)) and/or positively influencing hydration of the horny layer.

Advantageous moisturizers for the purposes of the present invention are, for example, glycerol, lactic acid, pyrrolidonecarboxylic acid and urea. It is also particularly advantageous to use polymeric moisturizers from the group of polysaccharides which are soluble in water and/or swellable in water and/or gellable using water. Particularly advantageous are, for example, hyaluronic acid and/or a fucose-rich polysaccharide which is listed in Chemical Abstracts under the registry number 178463-23-5 and is available, for example, under the name Fucogel® 1000 from SOLABIA S.A.

The amounts of cosmetic or dermatological auxiliaries and carriers and perfume to be used is each case can easily be determined by the person skilled in the art by simple trial and error depending on the type of product in question.

An additional content of antioxidants is generally preferred. According to the invention, favorable antioxidants which can be used are any antioxidants suitable or conventional for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. μmol to pmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of gum benzoin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients, which are suitable according to the invention.

The amount of abovementioned antioxidants (one or more compounds) in the emulsions is preferably 0.001 to 30% by weight, particularly preferable 0.05 to 20% by weight, in particular 0.1 to 10% by weight, based on the total weight of the preparation.

If vitamin E and/or derivatives thereof are the antioxidant or antioxidants, it is advantageous to choose the respective concentrations thereof from the range from 0.001 to 10% by weight, based on the total weight of the formulation.

If vitamin A or vitamin A derivatives or carotenes or derivatives thereof are the antioxidant or the antioxidants, it is advantageous to choose the respective concentrations thereof from the range from 0.001 to 10% by weight, based on the total weight of the formulation.

The lipid phase can advantageously be chosen from the following group of substances:

mineral oils, mineral waxes

oils, such as triglycerides of capric or of caprylic acid, and also natural oils, such as, for example, castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alkyl benzoates;

silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lypodispersions is advantageously chosen from the group of esters of saturated and/or unsaturated, branched and/or unbranched alkanecarboxylic acids with a chain length of from 3 to 30 C atoms and saturated and/or unsaturated, branched and/or unbranched alcohols with a chain length of from 3 to 30 C atoms, and from the group of esters of aromatic carboxylic acids and saturated and/or unsaturated, branched and/or unbranched alcohols with a chain length of from 3 to 30 C atoms. Such ester oils can then be advantageously chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyl-decyl stearate, 2-octyldodecyl palmitate, oleyl oleate, oleyl erucate, erucyl oleate, erucyl erucate, and synthetic, semisynthetic and natural mixtures of such esters such as, for example, jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and of fatty acid triglycerides, namely the triglycerol esters of saturated and/or unsaturated, branched and/or unbranched alkanecarboxylic acids having a chain length of from 8 to 24, in particular 12 to 18, carbon atoms. The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyidodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, for the purposes of the present invention, paraffin oil, squalane and squalene are to be used advantageously.

The oil phase can also advantageously have a content of cyclic or linear silicone oils, or consist entirely of such oils, although it is preferred to use an additional content of other oil phase components apart from the silicone oil or the silicone oils.

Cyclomethicone (octamethylcyclotetrasiloxane) is advantageously used as silicone oil to be used according to the invention. However, other silicone oils are also to be used advantageously for the purposes of the present invention, for example hexamethylcyclotrisiloxane, polydimethylsiloxane, poly(methylphenylsiloxane).

Also particularly advantageous are mixtures of cyclomethicone and isotridecyl isononanoate, and of cyclomethicone and 2-ethylhexyl isostearate.

The aqueous phase of the formulations according to the invention may advantageously comprise

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and, in particular, one or more thickeners which can be chosen advantageously from the group consisting of silicon dioxide, aluminum silicates, polysaccharides and derivatives thereof, e.g. xanthan gum and/or hydroxypropylmethylcellulose, in each case individually or in combination.

The examples below serve to illustrate the present invention without limiting it. Unless stated otherwise, all amounts, fractions and percentages are based on the weight and the total amount or on the total weight of the preparations.

### EXAMPLES

| | EXAMPLE 1 |
|---|---|
| | % by wt. |
| Chitosan | 1.0 |
| Lecithin | 1.0 |
| Glycerol | 3.0 |
| Lactic acid (90% strength aqueous solution)¹ | 0.6 |
| Perfume, preservative, dyes, | q.s. |
| Antioxidants etc. | |
| Ethanol | 35.0 |
| Water | ad 100% |
| PH | ca. 4.5 |

| | EXAMPLE 2 |
|---|---|
| | % by wt. |
| Chitosan | 0.1 |
| Lecithin | 3.0 |
| Glycerol | 15.0 |
| Lactic acid (90% strength aqueous solution)¹ | 0.06 |
| Ethanol | 70.0 |
| Perfume, preservative, dyes, | q.s. |
| Antioxidants etc., sodium hydroxide solution | |
| Water | ad 100% |
| PH | ca. 5.5 |

¹ It is also possible to use all other acids which produce soluble chitosan salts, such as, for example, phosphoric acid, acetic acid, ascorbic acid (under a protective gas), hydrochloric acid, glycolic acid, nitric acid, 2-pyrrolidone-5-carboxylic acid, malic acid, salicylic acid, benzoic acid, malonic acid, succinnic acid, adipic acid, glutaric acid and/or glutamic acid. Also, by changing the chitosan/acid ratio, it is possible to vary the pH within a range from 3.5 to 5.5.

| | EXAMPLE 3 |
|---|---|
| | % by wt. |
| Chitosan | 3.0 |
| Lecithin | 1.5 |
| Propylene glycol | 5.0 |
| Lactic acid (90% strength aqueous solution)¹ | 0.6 |
| Ethanol | 17.5 |
| Isopropanol | 7.5 |
| Perfume, preservative, dyes, | q.s. |
| Antioxidants etc. | |
| Water | ad 100% |
| PH | ca. 4.5 |

| | EXAMPLE 4 |
|---|---|
| | % by wt. |
| Chitosan | 1.0 |
| Lecithin | 2.0 |
| Glycerol | 20.0 |
| Lactic acid (90% strength aqueous solution)¹ | 0.6 |
| Perfume, preservative, dyes, | q.s. |
| Antioxidants etc. | |
| Cyclomethicone | 50.0 |
| Polysorbate ® 20 (Uniqema)² | 2.0 |
| Water | ad 100% |
| PH | ca. 4.5 |

² Solubility promoter with an HLB value greater than 15

| | EXAMPLE 5 |
|---|---|
| | % by wt. |
| Chitosan | 1.0 |
| Lecithin | 1.0 |
| Glycerol | 20.0 |
| Lactic acid (90% strength aqueous solution)¹ | 0.6 |
| Perfume, preservative, dyes, | q.s. |
| antioxidants etc. | |
| Isopentane | 10.0 |
| Water | ad 100% |
| PH | ca. 4.5 |

| | EXAMPLE 6 |
|---|---|
| | % by wt. |
| Chitosan | 1.5 |
| Lecithin | 2.5 |
| Glycerol | 20.0 |
| Lactic acid (90% strength aqueous solution)¹ | 0.6 |
| Perfume, preservative, dyes, | q.s. |
| antioxidants etc. | |
| Ethanol | 30.0 |
| Dimethicones | 5.0 |
| Cyclomethicones | 15.0 |
| PH | ca. 4.5 |

### APPENDIX

Title

COSMETIC OR DERMATOLOGICAL FORMULATIONS FOR THE CARE AND COOLING OF THE SKIN AFTER SUNBATHING

List of Inventors

1. Rainer KRÖPKE Nationality: German Achtemdiek 23 22869 Schenefeld Germany

2. Brois SYSKOWSKI Nationality: German Kaemmererufer 12 b 22303 Hamburg Germany

3. Birgit GROTELÜSCHEN Nationality: German König-Heinrich-Weg 57 22459 Hamburg Germany

Claim to Priority

Priority is hereby claimed under 35 USC 119 on the basis of German Application No. 100 36 051.3, filed on Jul. 25, 2000.

Assignment

This application shall be effected in the name of Beiersdorf Aktiengesellschaft. “Express Mail”Mailing Label No. EL 851060654 US Date of Deposit: Jul. 17, 2001 NORRIS, McLAUGHLIN & MARCUS By:

1. A cosmetic or dermatological formulation with a content of
a) at least one volatile substance which has an evaporation number of less than 30,
b) at least one chitosan and
c) at least one lecithin.

2. The cosmetic or dermatological formulation as claimed in claim 1, which additionally comprises active ingredients chosen from the group of anti-inflammatory substances.

3. The cosmetic or dermatological formulation as claimed in either of claims 1 or 2, which additionally comprises refatting agents and humectants.

4. The cosmetic or dermatological formulation as claimed in any of claims 1 to 3, which is in the form of a multiple emulsion, microemulsion, Pickering emulsion or sprayable emulsion.

5. The cosmetic or dermatological formulation as claimed in any of the preceding claims, wherein the evaporation number of the volatile substance(s) is between 1 and 20.

6. The use of a cosmetic or dermatological formulation as claimed in any of claims 1 to 4 for the care of photodamaged skin and/or for the alleviation of the secondary reactions of the skin to the effect of UV radiation.

7. The use of
a) at least one chitosan and
b) at least one lecithin in a cosmetic or dermatological formulation for delaying the evaporation of volatile substances which have an evaporation number of less than 30, following application of the formulation to the skin or the scalp.

8. A cosmetic or dermatological sunscreen set, comprising in each case at least one cosmetic or dermatological formulation A and B, wherein
a) formulation A comprises at least one filter substance which filters UV-A and/or UV-B rays,
b) formulation B comprises
i) at least one volatile substance which has an evaporation number of less than 30,
ii) at least one chitosan and
iii) at least one lecithin, where
c) the formulations A and B are physically separate from one another and can be removed from a multicompartment package separately from one another.
